(19) United States
(12) Patent Application Publication
(10) Pub. No.: US 2001/0056230 A1
(43) Pub. Date: Dec. 27, 2001

(54) Computer-aided apparatus and method for preoperatively assessing anatomical fit of a cardiac assist device within a chest cavity

(76) Inventors: Jacob H. Barak, Oranit (IL); Thomas J. Walsh, Boston, MA (US)

Correspondence Address: Patrick J. O'Shea, Esq., Samuels, Gauthier & Stevens, LLP, Suite 3300, 225 Franklin Street, Boston, MA, 02110, US

(21) Appl. No.: 09/726,635

(22) Filed: Nov. 30, 2000

Related U.S. Application Data
(60) Provisional application No. 60/168,004, filed on Nov. 30, 1999.

(51) Int. Cl. A61B 5/05

(52) U.S. Cl. 600/407; 382/128; 600/411; 600/427

(57) ABSTRACT

A computerized modeling tool that is used to form displayable images, which allow a user to assess the fit of a cardiac assist device within a prospective surgical patient's chest cavity. The computerized modeling tool includes a processing device that receives (i) a plurality of two-dimensional cross sectional digitized images representative of the prospective patient's chest cavity, and (ii) at least one data file representative of a three-dimensional model of the exterior of the cardiac assist device. The processing device processes this information to form a first composite displayable image of the cardiac assist device positioned within the chest cavity with selected anatomical segments displayed in uniquely associated colors presented on a display, to allow a user to view the displayable image to assess possible positions of the cardiac assist device within the chest cavity. The present invention allows a user to accurately assess whether or not a cardiac assist device properly fits within the chest cavity of the candidate

### CROSS REFERENCE TO RELATED APPLICATIONS

The present application claims the benefit, under 35 U.S.C. §1 19(e), of U.S. Provisional Patent Application Serial No. 60/168,004 filed Nov. 30, 1999, entitled APPARATUS AND METHOD FOR ASSESSING SPATIAL ORIENTATION AND ALIGNMENT OF A CARDIAC ASSIST DEVICE WITHIN A CHEST CAVITY, and U.S. Provisional Patent Application (serial number unknown) filed Nov. 29, 2000, which applications are hereby incorporated herein by reference.

### BACKGROUND OF THE INVENTION

### Field of Use

The present invention relates generally to cardiac assist devices and, more particularly, to a computer modeling approach for assessing preoperatively whether a cardiac assist device will fit within a patient's chest cavity.

### Related Art

A totally implantable artificial heart offers the potential for an excellent quality of life for the recipient. Recent progress in modem technology, improvements in surgical techniques and increased understanding of circulatory physiology of cardiac assist device recipients indicate that a permanent mechanical replacement heart is now becoming a viable therapy for the treatment of patients having end-stage heart failure.

Realization of this potential requires minimization of the size and weight of the implantable elements including the blood pump assembly. Current design activities have focused on the most effective, anatomically compatible configuration of the blood pump, including the inflow and outflow ports. However, because the size, shape and topography of the anatomical structures of the chest cavity vary among patients, a particular blood pump will not fit into the chest cavity of all candidate patients.

Conventionally, surgical teams determined whether a candidate patient could receive a temporary cardiac assist device simply by performing stemotomy or other surgical procedure and comparing the physical dimension of the patient's chest cavity with the device. Recent advances in imaging technology have made available X-ray, MRI and/or CT images of the patient's anatomy. In an effort to avoid unnecessary surgery, such images of a patient's chest cavity are routinely reviewed before implanting a cardiac assist device. A similar approach can be used to make a determination as to anatomical fit of a total artificial heart device prior to surgery. Although viewing such images will likely result in an accurate decision for some candidate patients, its accuracy is quite limited. There are a host of patients which can be incorrectly accepted due to slight variations in anatomical structures that prevent the replacement heart device from fitting into the chest cavity. Although such variations can be accommodated during implantation of a cardiac assist device, there is less flexibility with a total replacement device. Therefore, there is a need for a reliable technique for determining preoperatively the anatomical fit of a total artificial heart device in a patient's chest cavity.

### SUMMARY OF THE INVENTION

The present invention is directed to a computerized modeling tool that generates displayable images of a cardiac assist device and a patient's chest cavity based on digitized images representative of the patient's chest cavity and at least one data file representative of a three-dimensional model of the exterior of the cardiac assist device. The cardiac device as well as individual anatomical segments can be graphically manipulated independently to enable a surgeon to determine preoperatively whether the cardiac device can fit with the patient's chest cavity, including determining the proper relative position and orientation of the device and anatomical segments. In addition, to insuring proper fit and alignment with the circulatory system, the present invention enables the surgeon to anticipate preoperatively the additional surgical techniques, if any, that will be needed to be performed to implant the device.

Briefly, according to an aspect of the present invention, a computerized modeling tool is used to form displayable images that allow a user to assess the fit of a cardiac assist device within a prospective surgical patient's chest cavity. The computerized modeling tool includes a processing device that receives (i) a plurality of digitized images representative of the prospective patient's chest cavity, and (ii) at least one data file representative of a three-dimensional model of the exterior of the cardiac assist device. The processing device processes this information to form a first composite displayable image of the cardiac assist device positioned within the chest cavity with selected anatomical segments displayed on a display, to allow a user to view the displayable image to determine if the cardiac assist device fits within the prospective patient's chest cavity.

The processing device responds to command signals from a user (e.g., a surgeon) to generate a second composite displayable image of the cardiac assist device repositioned within the chest cavity. The second composite displayable image is presented on the display to allow a user to consider a different orientation of the cardiac assist device repositioned within the chest cavity.

Advantageously, the present invention allows a user to accurately assess whether or not a cardiac assist device properly fits within the chest cavity of the candidate patient. Specifically, the user can view user selectable computer generated images of prospective device positions within the chest cavity. The user may rotate the views, view different cross sections or select other viewing options to determine a preferred spatial orientation of the cardiac assist device within the chest cavity. Significantly, the user can assess various positions of the cardiac assist device within the patient's chest prior to surgery. The present invention may also be used during surgery to guide/assist the surgeon in positioning the assist device.

These and other objects, features and advantages of the present invention will become apparent in light of the following detailed description of preferred embodiments thereof, as illustrated in the accompanying drawings.

### BRIEF DESCRIPTION OF THE DRAWINGS

FIG. 1 is a functional block diagram illustration of a computerized modeling system for assessing spatial orientation of a cardiac assist device within a prospective surgical patient's chest cavity;

FIGS. 2A-2B are perspective views of a cardiac assist device;

FIG. 3 is a flowchart illustration of functional steps performed by a software routine associated with the computerized modeling system of FIG. 1;

FIG. 4 is a first CT image of the prospective patient's chest cavity;

FIG. 5 is a second CT image of the prospective patient's chest cavity;

FIG. 6 is a pictorial illustration of a composite displayable image of the cardiac assist device image positioned within the chest cavity;

FIG. 7 is a pictorial illustration of a composite displayable image in the form of a three-dimensional rendering of the cardiac assist device with selected anatomical elements of the chest cavity;

FIG. 8 is a pictorial illustration of a composite displayable in the form of a three-dimensional rendering of the cardiac assist device improperly positioned with respect to selected anatomical elements;

FIG. 9 is a pictorial illustration of a composite displayable image in the form of a three-dimensional rendering of the cardiac assist device repositioned with respect to the selected anatomical elements of FIG. 8 to avoid the device positioning problem illustrated in FIG. 8;

FIG. 10 is a functional block diagram illustration of processing associated with the presented invention; and

FIG. 11 is a schematic block diagram of a computer aided design (CAD) system for generating a composite image(s) of the cardiac assist device operably positioned.

### DETAILED DESCRIPTION

The present invention is directed to a computerized modeling tool that generates displayable images of a cardiac assist device and a patient's chest cavity based on digitized images representative of the patient's chest cavity and at least one data file representative of a three-dimensional model of the exterior of the cardiac assist device. The cardiac device as well as individual anatomical segments can be graphically manipulated independently to enable a surgeon to determine preoperatively whether the cardiac device can fit with the patient's chest cavity, including determining the proper relative position and orientation of the device and anatomical segments. In addition to insuring proper fit and alignment with the circulatory system, the present invention enables the surgeon to anticipate preoperatively the additional surgical techniques, if any, that will be needed to be performed to implant the device.

In the following exemplary description, the cardiac assist device is a total artificial heart. It should be understood, however, that the term “cardiac assist device” is meant to refer to all circulatory assist devices, whether they are temporary or permanent replacement devices and whether they assist or replace one or both ventricles of the natural heart. Furthermore, some devices are not positioned in the chest cavity but adjacent to it. It should become apparent to those of ordinary skill in the art from the present disclosure that the present invention can be utilized to determine preoperatively whether such devices will fit within their intended body cavity.

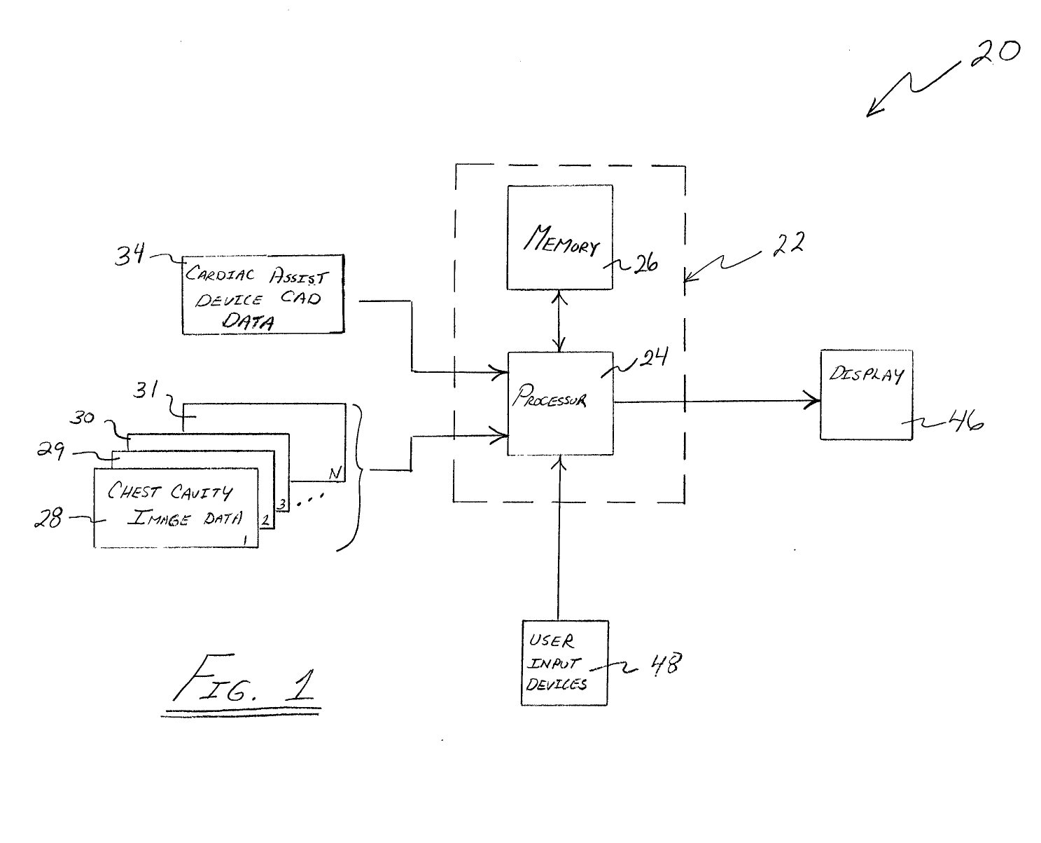

FIG. 1 is a functional block diagram illustration of a computerized modeling system 20 for assessing the fit of a cardiac assist device (not shown) within a prospective surgical patient's chest cavity. The system 20 includes a workstation 22 comprising a processor 24 and memory 26 (e.g., disk). In one embodiment the workstation is an IBM-compatible personal computer having at least one processor such as an Intel Pentium™ device.

The workstation 22 receives digitized image data 28-31 (e.g., two-dimensional cross sectional digitized images) representative of the prospective patient's chest cavity. The images may be input to the workstation through various known mechanisms such as a telemedicine device, from a diskette, tape or compact disk, or over a communications channel such as a telephone line, cable or wireless link. The images are preferably CT generated images of the prospective patient's chest cavity. MRI images may also be used.

The workstation 22 also receives cardiac assist device data 34 representative of a three-dimensional model of the exterior of the cardiac assist device. The device data 34 may be generated with known computer aided design (CAD) software tools, such as PROENGINEER™ available from Parametric Technologies Corporation, or AUTOCAD™. This data is input to the workstation via known techniques, as set forth for example in the preceding paragraph.

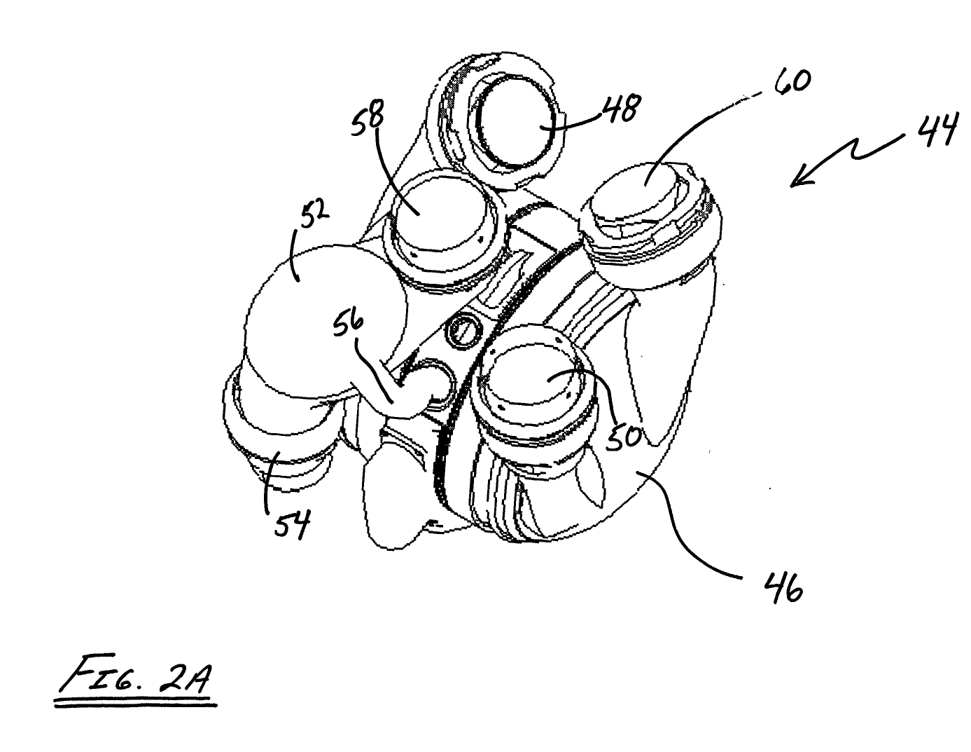

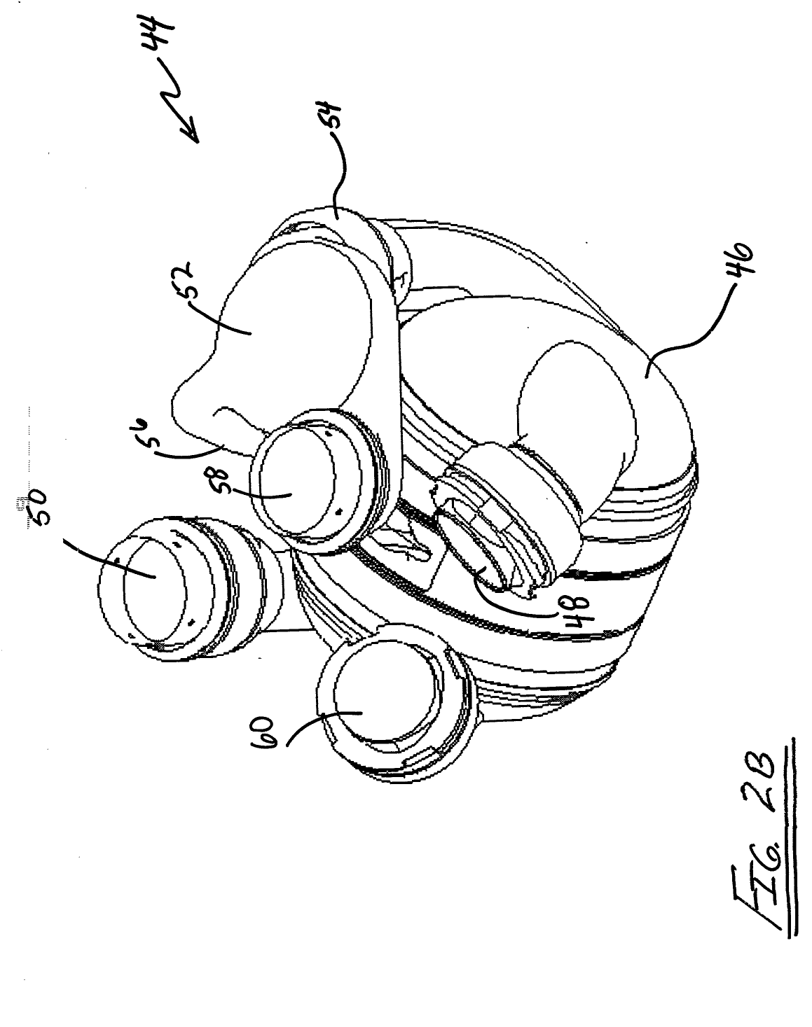

FIGS. 2A-2B are perspective views of a cardiac assist device 44. This device is a chest implantable replacement heart. Referring to FIGS. 2A and 2B, the exterior surfaces of the device 44 include a housing 46, a left outlet 48 that connects to the aorta and a right inflow 50 that connects to the right atrium. The exterior surfaces also include a compliance chamber 52, a valve housing 54, a fluid flow line 56, an inlet 58 from the left atrium and a right outlet 60 to the pulmonary artery.

Referring again to FIG. 1, the processor 24 processes the chest cavity data images 28-31 and the cardiac assist device data 34 to form a composite image (e.g., a bit mapped image) of the cardiac assist device operably positioned within the chest cavity. The composite image is presented to a user (e.g., a surgeon) on a display 46. The workstation 20 also includes user input devices 48, which may include a computer keyboard, a computer mouse, or other conventional devices to input commands in order to manipulate the image(s) presented on the display 46.

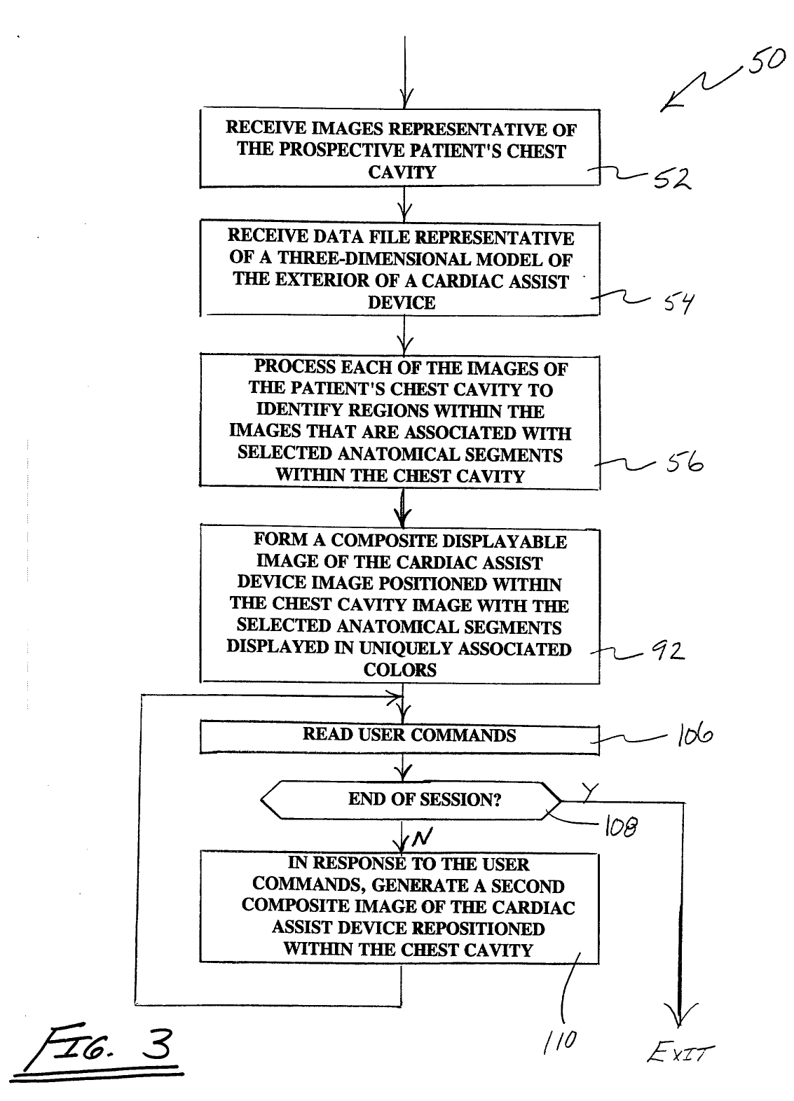

FIG. 3 is a flowchart illustration of functional steps performed by an executable software routine 50 that is stored in the memory device 26 (FIG. 1) and executed by the processor 24 (FIG. 1) to form the composite image. The routine 50 includes a step 52 to receive the images 28-31 (FIG. 1) representative of the prospective patient's chest cavity. Step 54 is performed to receive the device data 34 (FIG. 1).

Step 56 is executed to process each of the images 28-31 (FIG. 1) of the patient's chest cavity to identify regions within these images that are associated with selected chest cavity anatomical parts. The selected anatomical segments may include image segments indicative of the patient's pulmonary arteries, lungs, aorta, diaphragm, esophagus, stomach, left and right ventricles, left and right atria, pulmonary veins, inferior and superior vena cavas, and the rib cage. In general, this step involves associating each of the spatially distinct segments within the images 28-31 (FIG. 1) with a chest cavity anatomical segment (e.g., the pulmonary arteries, lungs, aorta, etc). This step may be performed either manually or automatically.

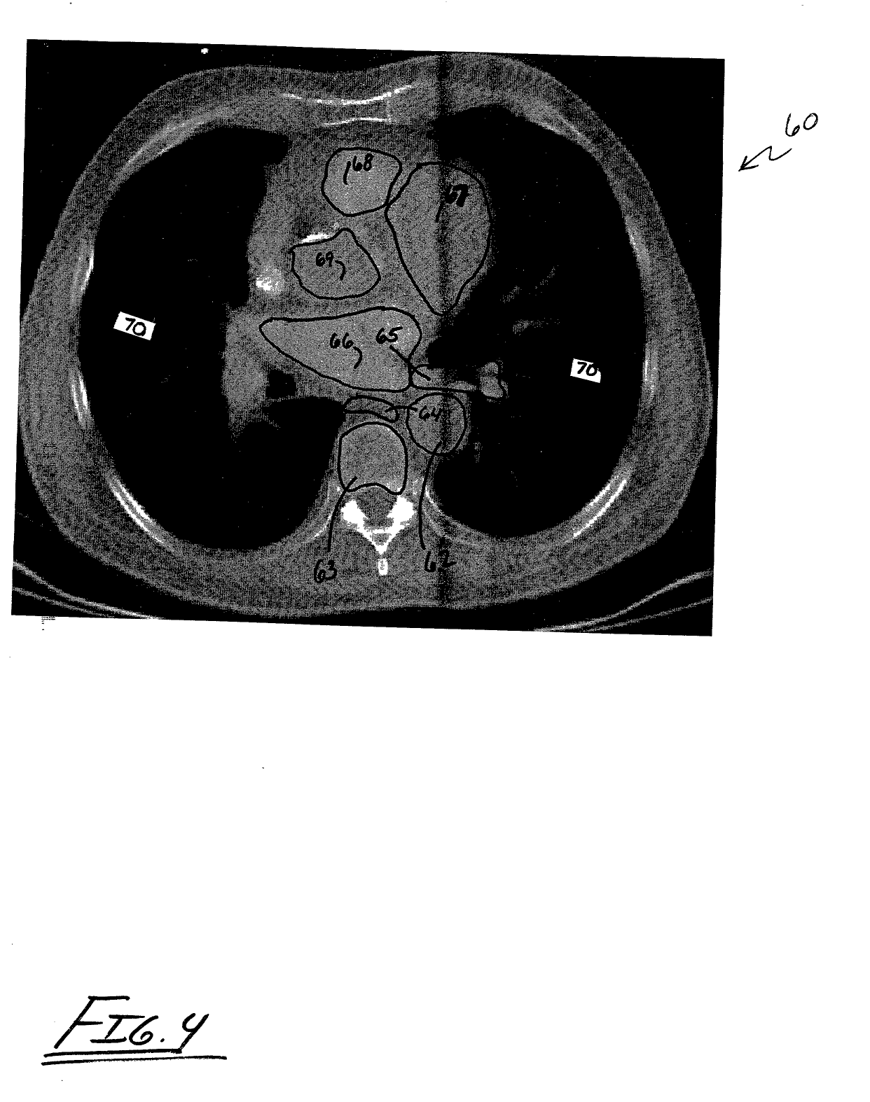

In one embodiment, step 56 may be performed in cooperation with the user who views each of the images and provides inputs regarding which anatomical segment is associated with various spatial regions of the image. FIG. 4 is a CT image 60 of the prospective patient's chest cavity. The CT image 60 includes various spatial regions that a trained user (e.g., a radiologist, a surgeon, etc) can identify as anatomical segments. For example, while viewing the image 60 the trained user can identify spatial region 62 as the descending aorta. Similarly, spatial region 63 is associated with the backbone. Table 1 specifies the associations between the various spatial regions in the CT image and the anatomical segments within the chest cavity.

TABLE 1

| ELEMENT # | ANATOMICAL SEGMENT |
|---|---|
| 62 | descending aorta |
| 63 | backbone |
| 64 | esophagus |
| 65 | pulmonary veins |
| 66 | left atrium |
| 67 | left ventricle |
| 68 | right ventricle |
| 69 | ascending aorta |
| 70 | lungs |

Various techniques may be used to perform the segmentation automatically. For example, the segmentation may be performed by image pixel thresholding. This technique involves setting gray scale threshold ranges for each of the anatomical segments within the chest cavity. The gray scale level intensity of each pixel in the image is then automatically compared to the threshold ranges to determine the range within which the pixel intensity falls, and thus the anatomical segment associated with the range. Editing may also be performed to manually select/edit the spatial regions of the image associated with the selected anatomical segments. The manual editing may be performed using a light pen, a template and other known pointing devices to select spatial regions within the CT image.

Once segmentation of the first CT image 28 (FIG. 1) has been completed, the task is performed again for the second CT image 29 (FIG. 1), an example of which is illustrated in

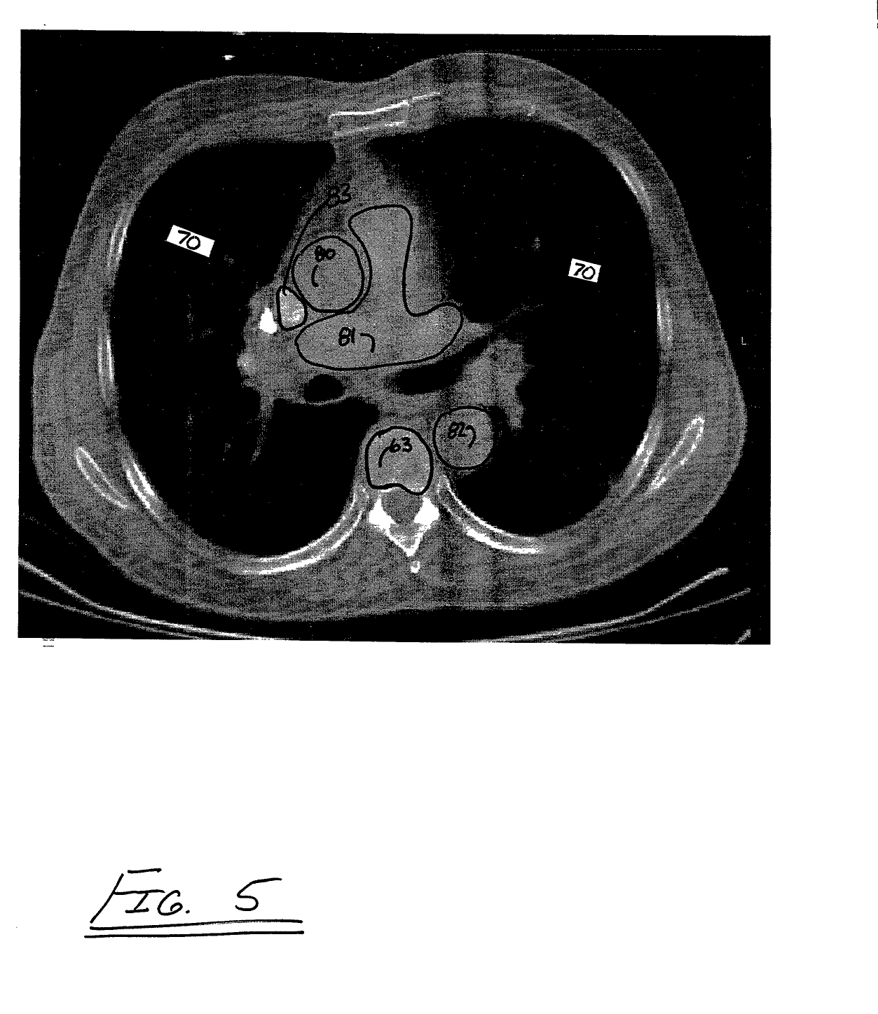

FIG. 5. Table 2 specifies the associations between the various spatial regions in the second CT image and the anatomical segments within the chest cavity that are easily identified in FIG. 4.

TABLE 2

| ELEMENT # | ANATOMICAL SEGMENT |
|---|---|
| 80 | ascending aorta |
| 81 | pulmonary veins |
| 82 | descending aorta |

Referring again to FIG. 3, the step 56 is performed for each of the CT images representative of the prospective patient's chest cavity. A complete set of CT images for a chest cavity may total N (e.g., fifty) images. Therefore, the step 56 is performed for each of the N number of CT images.

Step 92 is performed next to form a composite displayable image (e.g., a bit mapped image) of the cardiac assist device image positioned within the chest cavity, with the various selected anatomical segments displayed in uniquely associated colors. This step creates the composite image by processing: (i) the assist device data received in step 54 and (ii) the segmentation information and associations specified in step 56.

In a preferred embodiment, a tool for performing steps 52, 54, 56 and 92 is the commercially available executable software routine MIMICS™ available from Materialise Software, Inc. (www.materialise.be). This software routine is executed by the processor 24 (FIG. 1) and provides an interactive tool for visualizing and segmenting the CT images. This routine also generates three-dimensional renderings. MIMICS™ is a general purpose segmentation program for gray scale value images capable of performing step 56. In addition, this executable program processes any number of two-dimensional image slices. Of course, the number of slices is limited by the amount of workstation memory.

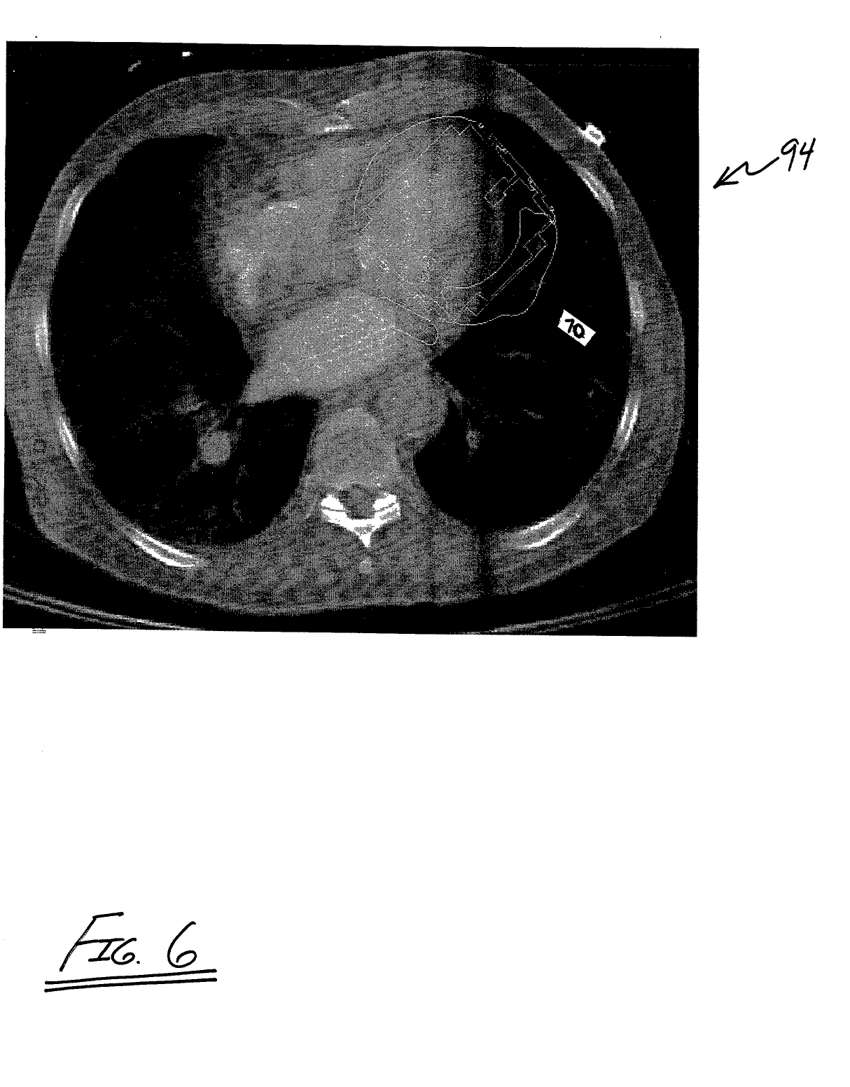

FIG. 6 is a pictorial illustration of a composite displayable image 94 of an outline of the cardiac assist device 44 image positioned within the chest cavity CT image. In this displayed image 94 the outline of the cardiac assist device is superimposed in an operable position onto the chest cavity image. This image may be used to assess the amount that the assist device 44 displaces the lung 70. For example, if the lung is displaced too much, then the assist device may need to be repositioned.

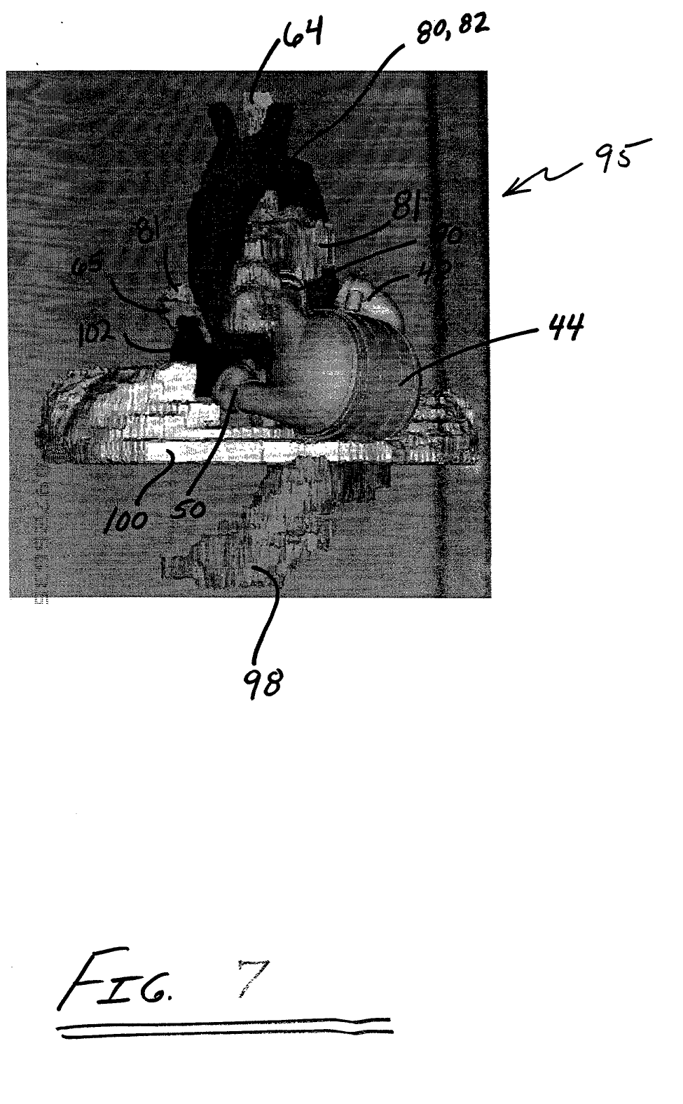

FIG. 7 is a pictorial illustration of a composite displayable image 95 in the form of a three-dimensional rendering of the cardiac assist device operably positioned with respect to selected chest cavity anatomical components. Notably, this rendering illustrates the pump inlet/outlets connected to their associated anatomical component. Specifically, the right inflow 50 is connected to the right atrium 102. Device right outflow 60 connects with the pulmonary arteries 81, while the left outflow 48 connects with the ascending aorta and the connections are preferably made by grafts (e.g., flexible tubing). This rendered image also presents an image of the patient's stomach 98 and diaphragm 100. The user may rotate this image, magnify it, add or delete anatomical segments, et cetera, by inputting commands to the workstation 20 (FIG. 1) via the user input devices 48 (FIG. 1).

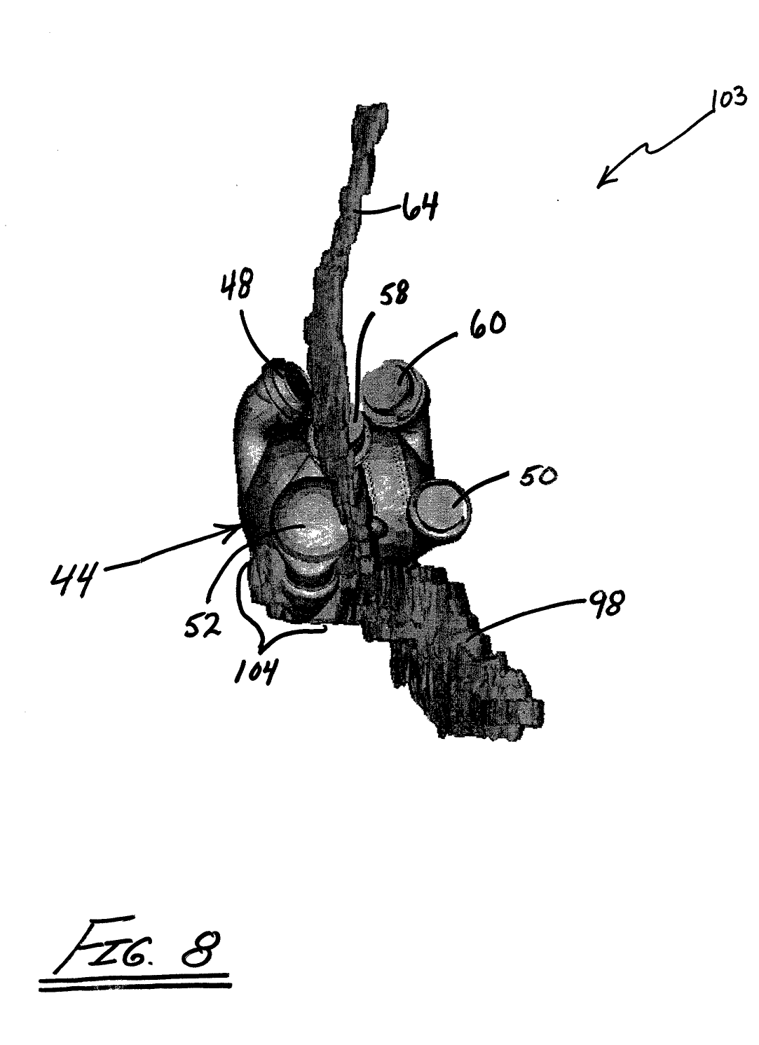

We shall now discuss an example of how the modeling tool of the present invention can alert a user to a possible problem prior to the implant surgery. FIG. 8 is a pictorial computer generated image 103 of the cardiac assist device and a portion of the patient's digestive tract including the stomach 98. Notably, in this image picture, the left inflow 58 passes directly through the junction of the esophagus and the stomach in area 104. Significantly, if the assist device 44 was placed into the chest cavity at this orientation and alignment, it would “pinch-off”/interfere with flow between the esophagus 64 and the stomach 98. As a result eating would be either very difficult or impossible. Advantageously, the surgeon can see this problem in this computer generate image, whereas he may not have seen the problem during the actual replacement procedure. The system may also automatically detect this interference and provide an audio/video annunciation to the user.

Referring again to FIG. 3, following the generation and display of an initial composite image of the assist device within the chest cavity, step 106 is executed to read user commands from the user input devices 48 (FIG. 1). Step 108 is then executed to determine whether or not the user has input a command to end the modeling session. If he has, the executable routine 50 is exited. Otherwise, step 110 is executed to generate a second composite image of the cardiac assist device repositioned within the chest cavity. For example, step 110 may respond to commands to reposition the assist device so it does not interfere with the flow path between the esophagus and the stomach. The location of the repositioning may be specified by the user, or the executable routine may suggest a new location for the assist device to prevent interfering with the flow path between the esophagus and the stomach. Alternatively, if the user commands are to display a slightly different view (e.g., a rotated view) of the device in the same position, step 110 will generate the new composite image in response to this user command. This step may also respond to user commands to remove or add an anatomical element to the image so the surgeon can further assess the fit of the assist device with or without that anatomical element in the image. For example, referring to FIGS. 7 and 8, to go from the image presented in FIG. 7 to the image presented in FIG. 8 the user issued commands to: (i) rotate the image, (ii) slightly reposition the assist device 44 and (iii) remove all the anatomical segments other than the esophagus 64 and the stomach 98. The images may be presented either alone on the display, side-by-side on the display or on different display devices.

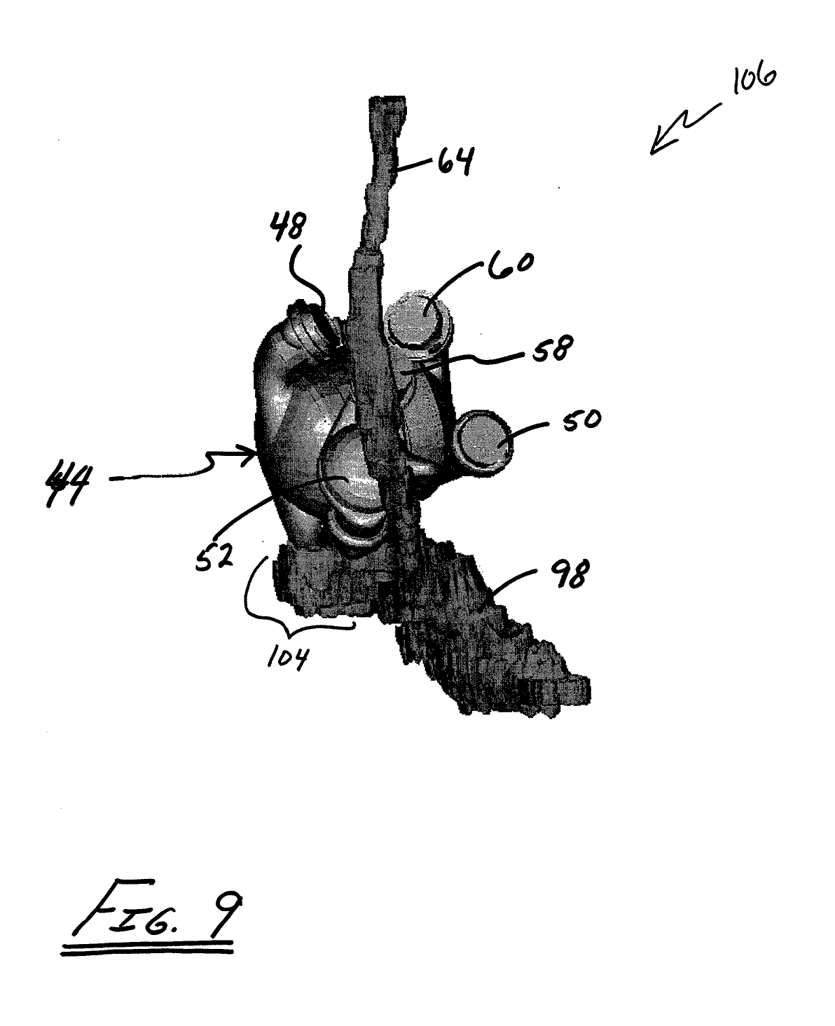

Referring now to FIGS. 3 and 8, to assess new positions for the assist device 44, the user inputs commands to reposition the assist device that are read by the step 106. Step 110 then generates a new composite image in response to those commands. FIG. 9 is a pictorial computer generated image 106 of the cardiac assist device 44 repositioned within the chest cavity in response to the user's commands. Notably, as shown in image 106, in its repositioned location the assist device 44 no longer compresses the junction between the esophagus 64 and the stomach 98 in the area of 104.

There are a number of fit criteria for assessing whether or not the position of the cardiac assist device within the chest is acceptable. First, the left and right inflows of the pump must be aligned with the left and right atria, respectively. The mitral valve is the junction between the left atrium and the natural left ventricle. The tricuspid valve is the junction of the right atrium and the natural right ventricle. Second, the assist device must be positioned within the rib cage. Third, no pulmonary vein may be compressed or pinched by the assist device. Other criteria include verifying that the descending aorta is not compressed; interference is minimized with the esophagus-gastric junction; and no interference with the esophagus or the diaphragm/stomach junction. One of ordinary skill will recognize that this is not an exhaustive list of factors to be assessed, but rather an abbreviated list in the interest of brevity. In addition, the factors to be considered will also depend on the mechanical properties (e.g., shape, weight, etc) of the assist device.

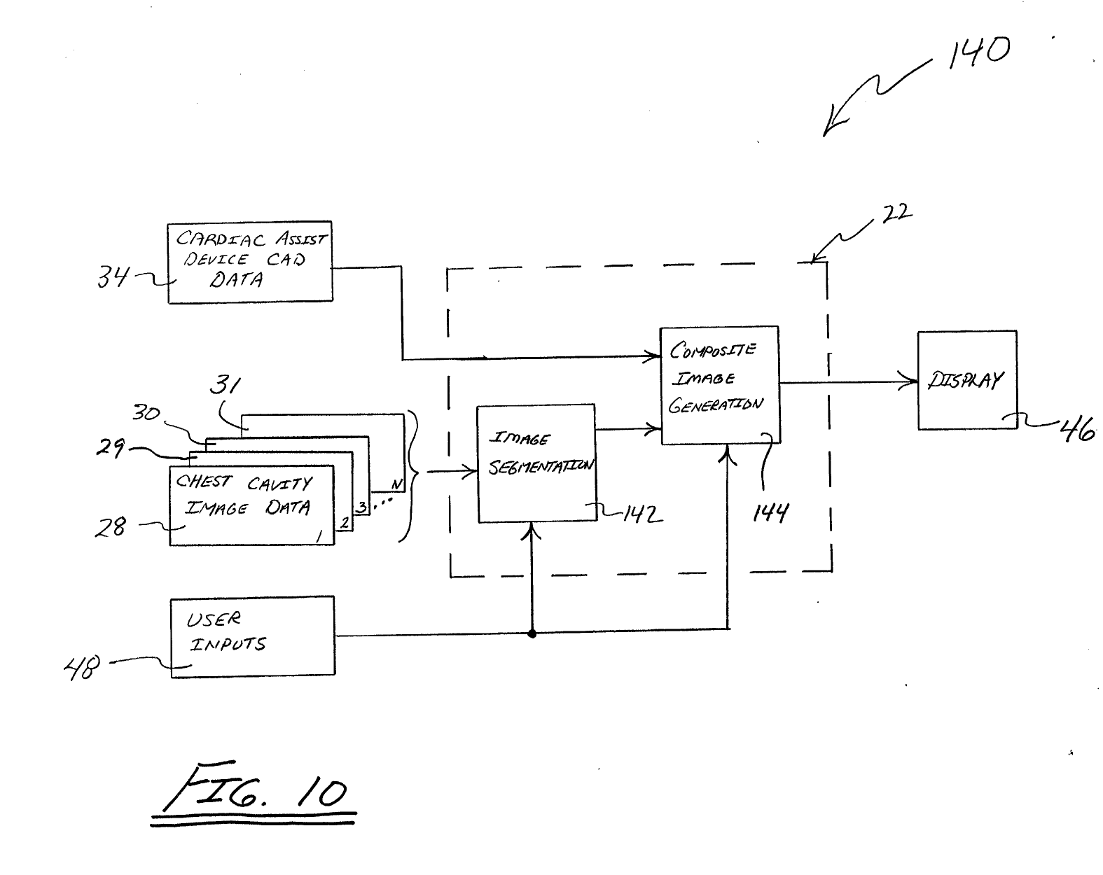

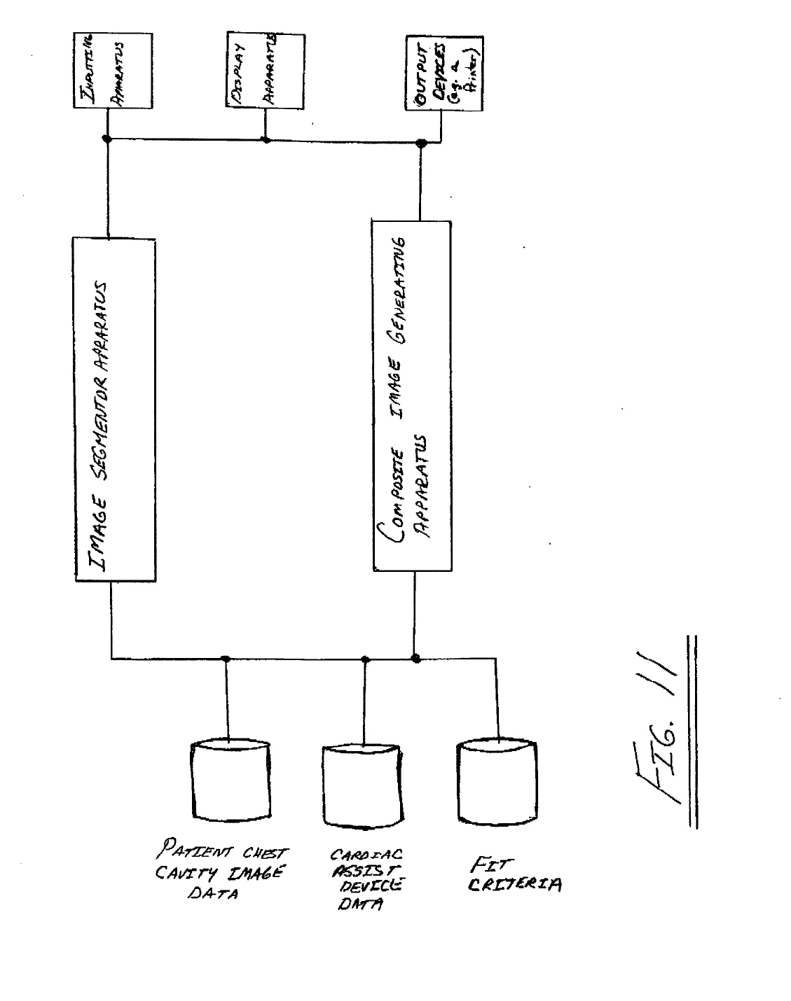

FIG. 10 is a functional block diagram illustration of processing 140 associated with the presented invention. As shown, the workstation 22 includes a segmentation function 142 and a composite image generation function 144. The segmentation function 142 receives the chest cavity images 28-31 and processes each of the images to identify regions within these images that are associated with selected chest cavity anatomical parts. The composite image generation receives the segment data and the cardiac assist device data and generates the composite images for presentation of the display 46. The details of these functions are discussed above. FIG. 11 is a schematic block diagram of a computer aided design (CAD) system for generating the composite image(s) of the cardiac assist device operably positioned. The composite image generating apparatus may be implemented in a workstation that includes the commercial off the shelf executable software routine MIMICS™ available from Materialise Software, Inc. (www.materialise.be), or a similar processing tool.

Advantageously, the modeling tool of the present invention assists in determining whether or not a patient is a candidate for the cardiac assist device. If it is determined that the patient is indeed a candidate (i.e., the cardiac assist device fits within the chest), the surgeon can plan the surgery more effectively using the present invention. For example, he can look at flowpath alignments from the front view (a view similar to the one during the surgery).

Although, not shown in the figure, on the display 46 (FIG. 1) the various chest cavity anatomical segments are preferably illustrated in uniquely associated colors for easier visualization. However, different gray scale may also be used for a monochrome display device.

It should be understood that the present invention is not limited to use with any particular computer platform, processor, or programming language. Aspects of the present invention may be implemented in software, hardware, firmware, or a combination of the three. The various elements of the system, either individually or in combination, may be implemented as a computer program product tangibly embodied in a machine-readable storage device for execution by a computer processor. Various steps of embodiments of the invention may be performed by a computer processor executing a program (i.e., software or firmware) tangibly embodied on a computer-readable medium to perform functions by operating on input and generating output. The computer-readable medium may be, for example, a memory in a computer or a transportable medium such as a compact disk, a floppy disk, or a diskette, such that a computer program embodying the aspects of the present invention can be loaded onto any computer. The computer program is not limited to any particular embodiment, and may, for example, be an application program, foreground or background process, driver, or any combination thereof, executing on a single computer processor or multiple computer processors. Computer programming languages suitable for implementing such a system include procedural programming languages, object-oriented programming languages, and combinations of the two.

While various embodiments of the present invention have been described above, it should be understood that they have been presented by way of example only, and not limitation. For example, it should be appreciated that the present invention may be implemented in other ways, and that the embodiments described herein are not limiting. It should be understood from this disclosure that the methods and techniques described herein with regard to the manner in which various components communicate and transfer data should not be construed as limiting, but merely one implementation of transferring the noted information. For example, a variable may be set in shared memory, a signal may be transmitted over a dedicated or shared line, or any one of a number of data bus techniques may be used. The implemented approach is a function of the selected embodiment. Thus, the breadth and scope of the present invention should not be limited by any of the above-described exemplary embodiments as various changes, omissions and additions to the form and detail thereof, may be made therein, without departing from the spirit and scope of the invention. Accordingly, the present invention should be defined only in accordance with the following claims and their equivalents.

What is claimed is:

1. A method of assessing the fit of a cardiac assist device within a prospective patient's chest cavity using a computerized modeling tool, the method comprising:
receiving a plurality of two-dimensional cross sectional digitized images representative of the prospective patient's chest cavity;
receiving at least one data file representative of a three-dimensional model of the exterior of a cardiac assist device;
processing each of said two-dimensional cross sectional digitized images to identify particular regions of each of said digitized images that are representative of selected anatomical segments within or proximate to the chest cavity; and
forming a composite displayable image of the cardiac assist device image and the chest cavity image with each said selected anatomical segment displayed so as to be distinguishable from neighboring anatomical segments.

2. The method of claim 1, wherein said anatomical segments include the pulmonary arteries, lungs, aorta, diaphragm, esophagus, stomach, left and right ventricles, left and right atria, pulmonary veins, inferior and superior vena cavas, and the rib cage.

3. The method of claim 1, wherein said step of receiving a plurality of two-dimensional cross sectional digitized images comprises the step of receiving a plurality of MRI images of the prospective patient's chest cavity.

4. The method of claim 1, wherein said step of receiving a plurality of two-dimensional cross sectional digitized images comprises the step of receiving a plurality of CT images of the prospective patient's chest cavity.

5. The method of claim 1, wherein said step of receiving a plurality of two-dimensional cross sectional digitized images comprises:
receiving a plurality of MRI images of the prospective patient's chest cavity; and
receiving a plurality of CT images of the prospective patient's chest cavity.

6. The method of claim 1, wherein said step of processing each of said two-dimensional cross sectional digitized images comprises the step of:
partitioning contiguous spatial regions of each of said two-dimensional cross sectional digitized images based upon image gray-scale levels to define said particular regions of said image associated with various anatomical segments.

7. The method of claim 6, wherein said step of partitioning comprises the step of:
partitioning to identify anatomical segments such as pulmonary arteries, lungs, aorta, diaphragm, esophagus, stomach, left and right ventricles, left and right atria, pulmonary veins, inferior and superior vena cavas, and the rib cage.

8. The method of claim 7, wherein said step of processing each of said two-dimensional cross sectional digitized images comprises the step of:
identifying mitral and tricuspid valves within the patient's chest cavity.

9. The method of claim 8, wherein the cardiac assist device comprises a pump housing having left and right inlets and associated left and right outlets, and said method further comprises the step of:
reprocessing, in response to user commands, said two-dimensional cross sectional digitized images to generate an image of the cardiac assist device repositioned within the chest cavity.

10. The method of claim 9, wherein said reprocessing step comprises the step of:
receiving user commands to reposition the cardiac assist device within the chest cavity to verify that the cardiac assist device does not improperly abut the ribcage with the cardiac assist device in the first operable position.

11. The method of claim 9, wherein said step reprocessing step comprises the step of:
receiving user commands to reposition the cardiac assist device image within the chest cavity image to verify that the cardiac assist device image does not improperly abut any anatomical segment of the patient.

12. The method of claim 11, wherein said reprocessing step comprises the step of:
repositioning, in response to user commands, the cardiac assist device image within the chest cavity image to allow a user to visually determine whether any circulatory vessels would be compressed by the cardiac assist device in the displayed position.

13. The method of claim 11, wherein said reprocessing step comprises the step of:
repositioning, in response to user commands, the cardiac assist device image within the chest cavity image; and
determining automatically whether any circulatory vessels are compressed by the cardiac assist device in the displayed position.

14. The method of claim 12, further comprising the steps of:
removing, in response to user commands, the image of one or more user-specified anatomical segments.

15. The method of claim 9, wherein said reprocessing step comprises the step of:
repositioning, in response to user commands, the cardiac assist device within the chest cavity to a first operable position such that the left and right inlets of the cardiac assist device are aligned with the mitral and tricuspid valves.

16. The method of claim 1, wherein said step of forming a composite displayable image of the cardiac assist device image within the chest cavity image with the selected anatomical segments comprises the step of:
forming said composite displayable image wherein the selected anatomical segments are displayed in uniquely associated colors.

17. The method of claim 1, wherein said step of forming a composite displayable image of the cardiac assist device image within the chest cavity image with the selected anatomical segments, comprises the step of:
forming said composite displayable image wherein the selected anatomical segments are displayed in uniquely associated gray scales.

18. The method of claim 1, wherein said step of forming a composite displayable image of the cardiac assist device image within the chest cavity image with the selected anatomical segments, comprises the step of:
forming said composite displayable image wherein the selected anatomical segments are displayed in uniquely associated shading.

19. An apparatus for assessing the fit of a cardiac assist device within a prospective patient's chest cavity, comprising:
an input port that receives (i) a plurality of two-dimensional cross sectional digitized images representative of the prospective patient's chest cavity, and (ii) at least one data file representative of a three-dimensional model of the exterior of the cardiac assist device;
means for processing each of said two-dimensional cross sectional digitized images to identify particular regions of each of said digitized images that are associated with selected anatomical segments within the chest cavity and for providing segment image data indicative thereof;
means for forming a first composite displayable image of the cardiac assist device image and the chest cavity image with the selected anatomical segments; and
a display that displays said first composite displayable image.

20. The apparatus of claim 19, further comprising:
means for receiving user commands and for providing user command signals indicative thereof;
wherein said means for processing responds to said user command signals to generate a second composite displayable image of the cardiac assist device repositioned within the chest cavity that is presented on said display, to allow a user to assess the fit of the cardiac assist device repositioned within the chest cavity of the prospective patient.

21. The apparatus of claim 19, wherein said means for forming said first composite displayable image comprises means for rendering the selected anatomical segments in uniquely associated colors to visually distinguish the selected anatomical segments.

22. The apparatus of claim 19, wherein said means for forming said first composite displayable image comprises means for rendering the selected anatomical segments in uniquely associated gray scales to visually distinguish the selected anatomical segments.

23. The apparatus of claim 19, wherein said means for forming said first composite displayable image comprises means for rendering the selected anatomical segments in uniquely associated shading to visually distinguish the selected anatomical segments.

24. The apparatus of claim 19, wherein said means for forming comprises a processor.

25. An apparatus for assessing the fit of a cardiac assist device within a prospective patient's chest cavity, comprising:
A) an input port that receives (i) a plurality of two-dimensional cross sectional digitized images representative of the prospective patient's chest cavity, and (ii) at least one data file representative of a three-dimensional model of the exterior of the cardiac assist device;
B) a processor, comprising
B1) means for processing each of said two-dimensional cross sectional digitized images to identify particular regions of each of said digitized images that are associated with selected anatomical segments within the chest cavity and for providing segment image data indicative thereof;
B2) means responsive to (i) said two-dimensional cross-sectional digitized images, (ii) said at least one data file, and (iii) said segment image data, for forming a first composite displayable image of the cardiac assist device image within the chest cavity image with the selected anatomical segments displayed in uniquely associated colors; and
C) a display that displays said first composite displayable image; and
D) means for receiving user commands and for providing user command signals indicative thereof;
wherein said means for processing responds to said user command signals to generate a second composite displayable image of the cardiac assist device repositioned within the chest cavity that is presented on said display, to allow a user to assess the fit of the cardiac assist device repositioned within the chest cavity of the prospective patient.

26. A system for assessing the fit of a cardiac assist device within a prospective patient's chest cavity, comprising:
an image segmentor that processes a plurality of digitized images representative of the prospective patient's chest cavity to identify particular regions of each of said digitized images that are associated with anatomical segments within the chest cavity, and provides segment data indicative of the location of the anatomical segments within said digitized images; and
a composite image generator that receives a data file representative of a three-dimensional model of the exterior of the cardiac assist device and said segment data, and forms a composite displayable image of the cardiac assist device image positioned within the chest cavity image with the anatomical segments.

27. The system of claim 26, wherein said digitized images comprise CT images.

28. The system of claim 26, wherein said digitized images comprise MRI images.

29. The system of claim 26, wherein said composite image generator includes means for automatically determining where to position said cardiac assist device within the chest cavity.

30. The system of claim 26, wherein said composite image generator is responsive to user command signals that specify where to position the image of said cardiac assist device within the chest cavity image.

31. The system of claim 29, wherein said means for automatically determining also includes means (i) for determining if left and right inflows to said cardiac assist device are aligned with patient left and right atria, respectively, (ii) for determining if said cardiac assist device is properly positioned with respect to the patient's rib cage, and (iii) for assessing if any pulmonary veins would be compressed by the position of said cardiac assist device.

32. The system of claim 26, wherein said image segmentor is responsive to user inputs that define the association of the anatomical segments within the chest cavity with the particular regions in each of said digitized images.

33. The system of claim 26, wherein said image segmentor comprises means, responsive to user inputs, for defining which anatomical segments within the chest cavity are associated with the particular spatial regions in each of said digitized images, wherein said user inputs include data indicative of a user selected region of said image.

34. The system of claim 33, wherein said data indicative of a user selected region of the image comprises bit map data.

35. The system of claim 33, wherein said means for defining is responsive to user inputs from a light pen, which define the spatial region in said digitized image that is associated with a selected anatomical component.

36. The system of claim 33, wherein said means for defining is responsive to user inputs from a pointing device, which define the spatial region in said digitized image that is associated with the selected anatomical component.

37. The system of claim 26, wherein said image segmentor comprises:
means, for automatically initially determining where to position said cardiac assist device within the chest cavity, for presenting an initial image on said display indicative of the spatial regions in said image that is associate with various selected anatomical components, and for providing automatic generated mapping data indicative of which spatial regions within said initial image are associated with which of the selected anatomical components; and
means, responsive to user inputs, for editing said automatic generated mapping data to provide said segment data.

38. The system of claim 37, wherein said user inputs include user controlled pointing device input signals that are generated by a pointing device that selects spatial regions of said initial image presented on said display.

39. The system of claim 37, further comprising means responsive to said segment data for automatically determine if there is any physical interference between the cardiac assist device and any of the anatomical components, and for providing an annunciation to the user if there is physical interference.

40. A method of assessing the fit of a cardiac assist device within a prospective patient's chest cavity, comprising:
initially processing a plurality of digitized images representative of the prospective patient's chest cavity to identify particular regions of each of the digitized images that are associated with anatomical segments within the chest cavity, and providing segment data indicative of the location of the anatomical segments within said digitized images;
receiving a data file representative of the exterior of the cardiac assist device; and
processing the segment data and the data file representative of exterior of the cardiac assist device, to form an initial composite image of the cardiac assist device operably positioned within the chest cavity based upon fit criteria.

41. The method of claim 40, further comprising
receiving at least one command signal to reposition the cardiac assist device within the chest cavity and forming a second composite image illustrating the cardiac assist device repositioned within the chest cavity; and
displaying the second composite image.

42. The method of claim 41, wherein the initial composite image and the second composite image are displayed side by side.

43. The method of claim 41, wherein the initial composite image and the second composite image are displayed on the same display.

44. The method of claim 40, wherein said step of processing comprises:
positioning the cardiac assist device with respect to the anatomical components within the initial composite image such that left and right inflows of the cardiac assist device are operably aligned with left and right atria anatomical components respectively.

45. The method of claim 44, wherein said step of processing comprises:
positioning the cardiac assist device respect to the anatomical components within the initial composite image such that the cardiac assist device is positioned within the rib cage anatomical components.

46. The method of claim 44, wherein said step of processing comprises:
positioning the cardiac assist device respect to the anatomical components within the initial composite image such that the cardiac assist device does not compress pulmonary veins anatomical components.

47. A method of forming a displayable image of a cardiac assist device positioned within a prospective patient's chest cavity, comprising:
receiving a bit mapped image indicative of the exterior of at least a portion of the cardiac assist device, and providing a first bit map indicative thereof;
receiving a bit mapped image indicative of at least a first portion of the prospective patient's chest cavity and at least one anatomical segment within the chest cavity, and providing a second bit map indicative thereof; and
processing said first and second bit maps to form a first displayable image.

48. The method of claim 47, comprising;
receiving a user command signal;
receiving a bit mapped image indicative of at least a second first portion of the prospective patient's chest cavity and said at least one anatomical segment within the chest cavity, and providing a third bit map indicative thereof; and
processing said first and third bit maps to form a second displayable image.

49. A method of manipulating a displayed image that includes an image of least a portion of a cardiac assist device and an image at least one anatomical segment associated with the chest cavity of a patient, comprising:
receiving a user command to reposition the image of the cardiac assist device with respect to the image of the anatomical segment display in the displayed image; and
processing a bit map image of the cardiac assist device and a bit map image of anatomical segment to reform the displayed image in response to said receiving a user command.
